(19) United States
(12) Patent Application Publication
(10) Pub. No.: US 2021/0318300 A1
(43) Pub. Date: Oct. 14, 2021

(54) OPTICAL BIOSENSOR COMPRISING DISPOSABLE DIAGNOSTIC MEMBRANE AND PERMANENT PHOTONIC SENSING DEVICE

(71) Applicant: UNIVERSITY OF ROCHESTER, Rochester, NY (US)

(72) Inventors: Benjamin L. MILLER, Penfield, NY (US); Michael BRYAN, Pittsford, NY (US); Daniel STEINER, Rochester, NY (US)

(21) Appl. No.: 17/268,727

(22) PCT Filed: Aug. 19, 2019
(86) PCT No.: PCT/US2019/046993

§ 371 Date: Feb. 16, 2021

Related U.S. Application Data
(60) Provisional application No. 62/719,499, filed on Aug. 17, 2018.

(51) Int. Cl. G01N 33/543; G01N 21/77

(52) U.S. Cl. CPC G01N 33/54373; G01N 21/77; G01N 2201/06113; G01N 2333/765; G01N 2333/59; G01N 2333/4737

(57) ABSTRACT

The present invention is directed to a biosensor (10) having a photonic sensing device (20), a sheet of a porous material (60), and an optically clear cover layer (70). The optically clear cover layer (70) may be removable and replaceable, whereby the sheet of porous material (60) can be replaced, and the photonic sensing device (20) can be re-used. Detection devices (810, 910) that include the biosensor (10), as well as methods of making and using the biosensor (10) are also disclosed.

This application claims the priority benefit of U.S. Provisional Patent Application Ser. No. 62/719,499, filed Aug. 17, 2018, which is hereby incorporated by reference in its entirety.

### FIELD OF USE

This disclosure relates to a biosensor, a detection device containing the biosensor, methods of detecting a biological molecule, and methods of making a biosensor.

### BACKGROUND

There is enormous interest in the use of paper-based diagnostics because of their versatility and low cost. It is very challenging, however, to implement quantitative diagnostic tests in a paper format, and analytical sensitivity is also a concern. In contrast, silicon photonic devices have been demonstrated to have remarkable sensitivity, while also enabling multiplex (multi-analyte) detection capability. Cost is a significant concern with silicon photonics.

By way of example, U.S. Pat. No. 7,019,847 to Bearman et al. (“Bearman”) describes a biosensor including a ring interferometer, one volumetric section of the ring interferometer being a sensing volume, a laser for supplying light to the ring interferometer, and a photodetector for receiving light from the interferometer. A sol gel containing capture molecules is deposited on top of the ring resonator that forms the ring interferometer. However, there is no indication in Bearman that the biosensor is reusable or that the sol gel may be removed and a new sol gel deposited. Thus, once the sol gel is used, or is incapable of regeneration, the entire biosensor is rendered unusable.

Bearman exemplifies a substantial deficiency in current integrated photonic sensor technology: the absence of a reliable system for pairing a very low cost, disposable membrane carrying capture molecules with a permanent or semi-permanent photonic sensor.

The present invention is directed to overcoming these and other deficiencies in the art.

### SUMMARY OF THE INVENTION

A first aspect relates to a biosensor that includes a photonic sensing device including a substrate and, formed on or in the substrate, a three dimensional structure suitable for producing an optical signal upon exposure to light; and a sheet of porous material covering the three dimensional structure suitable for producing an optical signal, where the sheet of porous material comprises one or more capture molecules and an optically clear cover layer connected to the photonic sensing device with the sheet of porous material between the cover layer and a portion of the photonic sensing device that contains the three dimensional structure.

A second aspect relates to a detection device that includes a biosensor as described herein, a light source that illuminates the photonic sensing device, and a photodetection device positioned to measure light emitted by the photonic sensing device.

A third aspect relates to a method of detecting a biological molecule. This method includes providing a biosensor as disclosed herein, introducing a liquid sample into contact with the sheet of porous material; and measuring a change in the light emitted by the photonic sensing device, where the change in the light emitted by the photonic sensing device indicates the binding of the biological molecule by the one or more capture molecules.

A fourth aspect relates to a method of making a biosensor. This method includes providing a photonic sensing device comprising a substrate and, formed on or in the substrate, a three dimensional structure suitable for producing an optical signal upon exposure to light; installing a sheet of porous material onto the substrate, where the sheet covers a portion of the photonic sensing device that contains the three dimensional structure for producing an optical signal, the sheet of porous material including one or more capture molecules; and installing an optically clear cover layer over the sheet of porous material, where the sheet of porous material is present between the cover layer and the portion of the photonic sensing device.

The present application demonstrates a diagnostic assay format that incorporates the best aspects of both paper diagnostics and silicon photonics by using a thin sheet of porous material, e.g., paper, as the carrier for reagents (e.g., specific biological capture molecules) while using a photonic chip as the biological sensor in a detection system.

The potential advantages of the described biological sensors include, but are not limited to, the following: (i) amenability to an arrayed design where several dozen assays may be performed simultaneously on a single device, (ii) detection of a range of different types of analytes, (iii) requirement for only a small sample volume, (iv) simplicity of operation preferably requiring only one sample-addition step, (v) delivery of a simple readout, (vi) re-usability of the more expensive photonic sensing device, (vii) use of a photonic sensing device with any of a variety of sheets of porous material (pre-loaded with one or more capture molecules), allowing for detection of an infinite number of target molecules with a single device, and (viii) depending on the sheet of porous material selected, methods known in the art for production of fluidic paths in paper (e.g., via wax transfer printing) allow for reconfigurable microfluidics on the sheet of porous material.

This brief summary has been provided so the nature of the invention may be understood quickly. Additional steps and/or different steps than those set forth in this summary may be used. A more complete understanding of the disclosed methods and products may be obtained by reference to the following description in connection with the attached drawings.

### BRIEF DESCRIPTION OF THE DRAWINGS

FIG. 1A is an exploded view of a biosensor 10 that includes a photonic chip with a ring resonator, a porous sheet, and an optically clear cover layer. FIG. 1B is a perspective view of the assembled biosensor.

FIG. 2A is an exploded view of a biosensor 110 that includes a photonic chip with a ring resonator, a porous sheet, an optically clear cover layer, and a clamping mechanism. FIG. 2B is a perspective view of the assembled biosensor 110.

FIG. 3A is an exploded view of a biosensor 210 that includes a photonic chip with a Mach-Zehnder interferometer, a porous sheet, and an optically clear cover layer.

FIG. 3B is a perspective view of the assembled biosensor 210.

FIG. 4A is an exploded view of a biosensor 310 that includes a photonic chip with a photonic crystal array, a porous sheet, and an optically clear cover layer. FIG. 2B is a perspective view of the assembled biosensor 310.

FIG. 5A is an exploded view of a biosensor 410 that includes a photonic chip with a porous sheet, and an optically clear cover layer that includes a diffraction grating.

FIG. 5B is a perspective view of the assembled biosensor 410.

FIG. 6A is an exploded view of a biosensor 510 that includes a photonic chip with an Archimedean whispering-gallery spiral waveguide, a porous sheet, and an optically clear cover layer. FIG. 2B is a perspective view of the assembled biosensor 510.

FIG. 7A is a side-elevational view of a biosensor 710 that includes a chip with a photonic element, a sheet of porous material, a cover, and a clamping mechanism. FIG. 7B is a side-elevational view of a detector 810 that includes biosensor, a light source, and a photodetection device. FIG. 7C is a side-elevational view of a detector 910 that includes biosensor having fiber optical cables to couple light from a light source into an inlet on the sensor chip as well as couple output light from the sensor chip to a photodetection device.

FIG. 8 is a schematic illustration of a biosensor 1010 that includes a photonic chip and a sheet of porous material. The left panel is an exploded view of the biosensor. The right panel is a perspective view of the assembled biosensor 1010.

FIGS. 9A-B are spectra collected for membranes saturated with nanopure water (left clusters) or sucrose solutions (right clusters). FIG. 9A shows that nanopure water spectra show clustered resonant wavelengths at 1550.75 nm and 5% sucrose at 1551.30 nm with an average resonant wavelength shift of 0.559 nm (σ=0.013 nm). FIG. 9B shows that nanopure water spectra show clustered resonant wavelengths at 1548.85 nm and 5% sucrose at 1549.45 nm with an average resonant wavelength shift of 0.662 nm (σ=0.013 nm).

FIG. 10 shows spectra of nitrocellulose membranes soaked in nanopure water (blue) and nitrocellulose membranes with 500 μg/ml α-CRP antibody with 1% BSA block in nanopure water (green). The resulting resonant wavelength shift is 0.06 nm.

FIG. 11 shows concentration-dependent changes in the resonant frequency when a strip of nitrocellulose is used to deliver protein solution to a ring resonator. 5 μl of BSA-spiked PBS was applied to a nitrocellulose strip. The graph (left panel) and spectra (right panel) show BSA relative resonance shifts, and the corresponding resonant wavelengths of the BSA-spiked PBS solutions, respectively.

FIG. 12 is a graph showing the resonant wavelength shift relative to air detected by individual ring resonators in a multi-ring resonator device. Two data points representing two separate measurements (FSR. Free Spectral Range) are shown for each ring.

### DETAILED DESCRIPTION

Unless defined otherwise, all technical and scientific terms used herein have the same meaning as is commonly understood by one of ordinary skill in the art to which the present disclosure belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The use of “including,” “comprising,” or “having” and variations thereof herein is meant to encompass the items listed thereafter and equivalents thereof as well as additional items. The use of any and all examples, or exemplary language (e.g., “such as”) is intended to better illuminate the embodiments and does not pose a limitation on the scope of the claims unless otherwise stated.

As used herein and in the appended claims, the singular forms “a,” “an,” and “the” include plural referents unless the context clearly dictates otherwise. Similarly, when the plural form is used it is to be construed to cover the singular form as the context permits. For example, “a” or “an” means “at least one” or “one or more.” Thus, reference to “an analyte” or “a biological molecule” refers to one or more analytes or biological molecules, and reference to “the method” includes reference to equivalent steps and methods disclosed herein and/or known to those skilled in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Wherever the word “about” is employed herein in the context of dimensions, amounts or concentrations, and coefficients of variation, it will be appreciated that such variables are approximate and as such may vary by ±10%, for example ±5% and preferably ±2% (e.g. ±1%) from the numbers specified herein.

As used herein, the term “analyte” or “target molecule” refers to a component of a sample which is desirably adsorbed (retained) and detected. The term can refer to a single component or a set of components in the sample. The analyte or target molecule may be, and in most cases is, a biological molecule.

One aspect relates to a biosensor that includes a photonic sensing device including a substrate and, formed on or in the substrate, a three-dimensional structure suitable for producing an optical signal upon exposure to light. The biosensor also comprises a sheet of porous material covering the three-dimensional structure suitable for producing an optical signal, where the sheet of porous material comprises one or more capture molecules and an optically clear cover layer connected to the photonic sensing device with the sheet of porous material between the cover layer and a portion of the photonic sensing device that contains the three-dimensional structure. Each of these components is discussed below.

In one embodiment, the photonic sensing device is a 2D photonic crystal array, a ring resonator, a Mach-Zehnder interferometer, a toroidal microcavity, a Bragg reflector, a diffraction grating, a plasmonic waveguide. Archimedean whispering-gallery spiral waveguides, or a nanoplasmonic pore.

The 2D photonic crystal array may have any suitable arrangement of pores formed in a substrate. One example of a 2D photonic crystal array is described in U.S. Application Publ. No. 2010/0279886 to Fauchet et al., the disclosure of which is incorporated herein by reference in its entirety. Photonic crystals (or crystal arrays) are an attractive sensing platform because they provide strong light confinement. These crystals can be designed to localize the electric field in the low refractive index region (e.g., air pores), which makes the sensors extremely sensitive to a small refractive index change produced by the capture of a targeted bio-molecule on the pore walls.

The ring resonator may have any suitable arrangement of ring features and working waveguide surfaces, including full, split, single, and/or multiple ring resonator constructions. One example of a ring resonator detector is described in PCT Publication WO 2013/053459, the disclosure of which is incorporated herein by reference in its entirety. A photonic sensing device of this type is very sensitive as a surface of the ring is scanned by an evanescent field of a light wave propagating within the ring. Currently, ring resonators are used to perform measurements with a selectively working absorber surface, which is labeled with one or more capture molecules and therefore plays an important role for an adequate specificity of the sensor. The capture of a targeted bio-molecule at the working surface causes the resonant condition of the ring to vary. Thus, an effective refractive index of the environment near the ring resonator changes upon capture of the targeted bio-molecule such that wavelengths of resonant modes are shifted. The detection of the shift into a coupled detection waveguide can indicate presence of the bio-molecule.

When the photonic sensing device comprises multiple ring resonators, each of the multiple ring resonators may be arranged in series on a single bus waveguide. In one embodiment, the photonic sensing device comprises a first ring resonator and a second ring resonator optically coupled to a bus waveguide. In another embodiment, the photonic sensing device comprises two or more ring resonators optically coupled to a bus waveguide. In yet another embodiment, the photonic sensing device comprises two or more bus waveguides each having two or more ring resonators optically coupled to the bus waveguides.

A waveguide is a structure which guides optical waves by total internal reflection (“TIR”). When a light beam traveling in a waveguide is totally internally reflected at the interface between the waveguide and an adjacent medium having a lower refractive index, a portion of the electromagnetic field of the TIR light penetrates shallowly into the adjacent medium. The use of waveguides in the design of biosensors has been described in numerous publications including U.S. Pat. No. 5,814,565 to Reichert et al., the disclosure of which is incorporated herein by reference in its entirety. The waveguide can be fabricated on a substrate surface. Alternatively, a waveguide can be formed within a recessed region of the substrate so as to form trenches on either side of the waveguide.

The construction and design considerations of Archimedean whispering-gallery spiral %% waveguides are described in Chen et al., “Design and Characterization of Whispering-gallery Spiral Waveguides,” Optics Express 22(5):5196, DOI:10.1364/OE.22.005196 (2014), which is hereby incorporated by reference in its entirety. A typical design includes two, interleaved Archimedean-shaped spirals: one that brings light from the exterior to the interior and the other that returns the light to the exterior. The interleaved Archimedean spirals are connected by an S-bend connection waveguide in the center to provide adiabatic change of mode location between clockwise and counterclockwise spiral waveguides. A change in the resonant response will occur upon target molecule binding, which changes the index of refraction outside the waveguide and thereby alters the resonant response.

Other waveguide-containing biosensors can also be utilized, including without limitation, slab waveguides of the type illustrated and described in U.S. Application Publ. No. 20180209910, planar waveguides of the type illustrated and described in U.S. Application Publ. No. 20180106724, and intersecting waveguide sensors of the type illustrated and described in U.S. Application Publ. No. 20180031476, the disclosures of which are incorporated herein by reference in their entirety.

Ultrahigh-Q silica toroidal microcavities can have any desired configuration, e.g., ring, ellipsoidal, or polygonal configurations. In one approach, an SiO₂ disk cavity can be fabricated on a silicon wafer by, e.g., thermal dioxidation, photolithography, and SiO₂ etching. The dioxide layer can be on the micron or submicron level. Next, the silicon sacrificial layer is undercut to form a Si post. With a combination of isotropic and anisotropic etching, a silicon post can be obtained and then the SiO₂ is exposed with a laser suitable to transfer the shape of the silicon post to the SiO₂ and form a smooth toroidal cavity of the desired configuration. As an alternative to SiO₂, other oxide glasses can be used to form the toroidal microcavity. The toroidal microcavity may have any suitable arrangement between the microcavity and working waveguide surfaces, including single or multiple microcavity constructions. Toroidal microcavities are useful to increase the distance between adjacent resonance wavelengths. One suitable structure of the microcavity sensor is described and illustrated in U.S. Application Publ. No. 20090097031 A1 to Armani et al., the disclosure of which is incorporated herein by reference in its entirety. One example for use of toroidal microcavities in a biosensor is described and illustrated in U.S. Application Publication No. 20090093375, the disclosure of which is incorporated herein by reference in its entirety.

A Bragg reflector is a sensor element utilizing more than one layer of materials with varying refractive indexes that result in detection of a reflectivity shift having one or more sharply defined luminescent peaks. A biosensor comprising a Bragg reflector is described in U.S. Pat. No. 7,226,733 to Chan et al., the disclosure of which is incorporated herein by reference in its entirety. The periodicity and design of the upper and lower Bragg reflectors can have any suitable configuration. When used with macroporous or mesoporous Bragg structures, it is possible to confine capture molecule location to the pores of the Bragg structures. Confinement to the pores rather that the outer surface of the Bragg structure can be achieved by masking the outer surfaces with the hydrogel particles prior to capture molecule coupling.

A diffraction grating operates at a fixed wavelength and detection angle by exploiting the variation in diffraction efficiency that occurs due to the presence of a chemical or biological species on a diffraction grating. Any of a variety of suitable diffraction grating structures (channel depth, width, and spacing) can be employed. In traditional diffraction-based biosensors, chemical or biological species are selectively adsorbed onto the top surface of a diffraction grating, giving rise to an increase in the diffraction efficiency proportional to the change in the grating thickness. Diffraction gratings may be ruled diffraction gratings, which comprise a series of grooves that have been ruled into the surface of the substrate. One exemplary diffraction grating based sensor device is described in U.S. Pat. No. 8,349,617 to Weiss et al., the disclosure of which is incorporated herein by reference in its entirety. Another exemplary diffraction grating sensor device is described and illustrated in U.S. Application Publ. No. 20180073987, the disclosure of which is incorporated herein by reference in its entirety.

A plasmonic waveguide involves excitations which do not exhibit the disadvantages associated with using light sources to determine a specific binding event. These surface plasmon polaritons or plasmonic mode excitations, i.e., electromagnetic excitations at a metal-dielectric interface, may be guided using structures that are much smaller than the wavelength of photons of the same frequency. Any of a variety of surface plasmon resonance (“SPR”)-biosensor structures can be utilized in forming the biosensor as described herein. These structures can be provided with any of a variety of topographical structures on the sensing surface. One exemplary plasmonic waveguide is described in U.S. Pat. No. 6,373,577 to Bräuer et al., the disclosure of which is incorporated herein by reference in its entirety. Another exemplary plasmonic waveguide is illustrated and described in U.S. Application Publ. No. 20170090077, the disclosure of which is incorporated herein by reference in its entirety.

Nanoplasmonic pores have the advantage of exhibiting unique optical transmission characteristics at resonant wavelengths. Any sensor structure comprising nanoplasmonic pores can be used in the present invention. The nanopores are formed in a submicron membrane including a metal film (e.g., gold, silver, platinum). The nanopores can be dimensioned to facilitate maximal response in consideration of the target molecule, but typically the nanopores are on the order of less than 250 nm, preferably less than 150 nm in diameter. Capture molecules bound within the nanopore features allow for specific binding of the target molecule within the nanopore structures. By monitoring the temporal variation in the plasmon resonance of the structure, flow-through nanoplasmonic sensing of specific biorecognition events (i.e., detection of the target molecule) can be achieved quickly in a low-volume flow through device. Exemplary nanoplasmonic biosensors are disclosed in U.S. Patent Publication No. 20120218550 to O'Mahony; and Jonsson et al., “Locally Functionalized Short-range Ordered Nanoplasmonic Pores for Bioanalytical Sensing,” Anal. Chem. 82(5):2087-94 (2010), the disclosures of which are incorporated herein by reference in their entirety.

It should be appreciated by those of ordinary skill in the art that any of a variety of substrates can be employed in the present invention. Substrates can be formed using any of a variety of materials. Exemplary materials include, without limitation, silicon such as crystalline silicon, amorphous silicon, or single crystal silicon, oxide glasses such as silicon dioxide, and polymers such as polystyrene.

The substrate may include one or more integral waveguides that afford an inlet for coupling light into, onto, or across the three-dimensional structure and an outlet for coupling light that passes from, through, or past the three-dimensional structure. There can be a single waveguide per three-dimensional structure, or more than one waveguide per three-dimensional structure. The construction of waveguides integral with the substrate are well known in the art.

The sheet of porous material may be formed of any suitable material that is sufficiently porous to allow, e.g., aqueous medium to move along or through the material. In certain embodiments, the porosity is also sufficient to allow target molecules and/or non-covalently tethered capture molecules to migrate through or across the material. Exemplary materials include, without limitation, polyethylene, polyethylene terephthalate, polyester, polypropylene, polytetrafluoroethylene (“PTFE”), polyvinyl fluoride, ethylvinyl acetate, polycarbonate, polycarbonate alloys, nylon, nylon 6, nylon 66, glass, polysaccharides, ceramics, thermoplastic polyurethane, polyethersulfone, polyvinylidene fluoride (“PVDF”), or derivatives thereof.

Suitable polysaccharides include, but are not limited to, cellulose or cellulose derivatives, e.g., cellulose acetate, cellulose acetate propionate, nitrocellulose, carboxymethyl cellulose, or dimethylamide of carboxymethyl cellulose. Additional suitable cellulose derivatives are described in U.S. Application Publ. No. 2012/0122691, which is hereby incorporated by reference in its entirety.

The sheet of porous material may be in the form of a paper or thin membrane. Specifically, the membrane may be glass fiber filter paper, cellulose filter paper, etc., commercially available from Sartorius, Millipore, Toyo Roshi, Whatman, etc. In one embodiment, the sheet of porous material is a PVDF membrane or a PTFE membrane. Synthetic membranes are also contemplated. See. e.g., Hansson et al., “Synthetic Microfluidic Paper: High Surface Area and High Porosity Polymer Micropillar Arrays,” Lab Chip 16(2):298-304 (2016), which is hereby incorporated by reference in its entirety

The sheet of porous material may be macroporous, mesoporous, or microporous. As used herein, the term “macroporous” refers to a matrix comprising defined pores which have diameters greater than 50 nm; the term “mesoporous” refers to a material comprising a matrix with defined pores which have diameters in intermediate range between 2 and 50 nm; and the term “microporous” refers to a matrix with defined pores which have diameters less than 2 nm.

The sheet of porous material can be any suitable thickness depending upon the intended use, but preferably less than about 180 microns, more preferably between about 100 to about 180 microns. In one embodiment, the paper is at least 100, 110, 120, 130, 140, 150, 160, or 170 microns thick. Typically, the thickness will vary inversely according to the desired porosity (i.e., higher porosity structures will be thicker than lower porosity structures) as well as according to the wavelength of light to be detected (i.e., structures which are used with shorter wavelength light can be thinner than structures which are used with longer wavelength light).

The sheet of porous material may comprise various zones that are positioned, at least partially, directly above the three-dimensional structure formed on or in the substrate of the photonic sensing device. By way of example, when the three-dimensional structure formed on or in the substrate of the photonic sensing device comprises one or more ring resonators, the sheet of porous material comprises one or more zones positioned, at least partially, directly above each of the one or more ring resonators.

In one embodiment, the sheet of porous material comprises a first zone comprising the one or more capture molecules and a second zone comprising a control capture molecule. In another embodiment, the sheet of porous material comprises (i) multiple test zones, where each test zone comprises one or more capture molecules, and (ii) one or more reference zones, where each reference zone comprises a control capture molecule. In this manner, the sheet of porous material can provide an array of sites (or “spots”) where capture molecules are located. Each spot may comprise any suitable concentration of one or more capture molecules that is optimized for detection, but typically nanomolar, micromolar, or picomolar amounts of the one or more capture molecules is present at each of the spots.

Methods of applying capture molecules to solid surfaces are well known in the art and include the use of contact and non-contact printing technologies. Suitable contact printing technologies include, e.g., solid pin printing, split pin printing, capillary printing, and micro-spot printing. Suitable non-contact printing technologies include, e.g., piezoelectric printing and syringe-solenoid printing. These same techniques can be used for applying one or more capture molecules to the sheet of porous material at the desired locations or zones.

In some embodiments, the sheet of porous material may be fabricated by coating paper layers with various substances using a printer, for example a laser or inkjet printer. The printer may be used to form a water-impermeable coating on the sheet of porous material. Toner or other substances generated by a printer may be used as a thermal adhesive to bond multiple layers of paper together in order to create 3D sheet of porous material.

As mentioned above, aspects of the present invention may be embodied using paper. Potential advantages of using paper include the following: paper is inexpensive, wicks fluids by capillary action, and may provide a large surface area for immobilizing and storing reagents.

If desired, the sheet of porous material may be fabricated by patterning paper into a network of hydrophilic channels and test zones bounded by hydrophobic barriers. The patterning process preferably defines the width and length of channels, and paper thickness preferably defines height and/or temporal aspects of the channel. This can be achieved, for example, by direct printing of hydrophobic and/or other substances onto paper. In particular, certain laser and/or inkjet printers can deposit and/or pre-deposit wax, gelatin, and/or other substances directly onto paper at low cost. Other techniques for deposition of the substances may be used.

For example, the design of the devices may be first prepared on a computer, the pattern may then be printed in wax, gelatin, and/or other substances onto paper using a commercially available printer, and the paper may then be heated to a temperature above the melting point of the material(s) so the material(s) reflows and creates hydrophobic barriers that span the thickness of the paper. Once a device is fabricated, reagents may be loaded onto the devices by applying solution(s) of reagent(s) onto the device and allowing related solvent(s) that carried the reagent(s) to evaporate.

In addition to patterning individual layers of paper, stacking multiple layers of patterned paper may be possible.

The available strategies for attaching the one or more capture molecules include, without limitation, covalently bonding a capture molecule to the sheet of the porous material, ionically associating the capture molecule with the sheet of the porous material, adsorbing the capture molecule onto the sheet of the porous material, or the like. In one embodiment, the one or more capture molecules are covalently attached to the sheet of the porous material. In accordance with this embodiment, the one or more capture molecules comprise a plurality of capture molecules covalently attached to the sheet of porous material at discrete locations.

The covalent attachment of capture molecules to paper and other thin membranes is known in the art. See, e.g., Kong et al., “Biomolecule Immobilization Techniques for Bioactive Paper,” Anal. Bioanal. Chem. 403:7-13, DOI:10.1007/s00216-012-5821-1 (2012); Su et al., “Adsorption and Covalent Coupling of ATP-Binding DNA Aptamers onto Cellulose,” Langmuir 23:1300-1302 (2007); Böhm et al., “Covalent attachment of enzymes to paper fibers for paper-based analytical devices,” Front. Chem. 6:214 (2018): Holstein et al., “Immobilizing affinity proteins to nitrocellulose: a toolbox for paper-based assay developers,” Anal. Bioanal. Chem. DOI 10.1007/s00216-015-9052-0 (2015), the disclosures of which are incorporated herein by reference in their entirety.

The optically clear cover may be formed of any suitable material, for example, glass, quartz, or plastics. In one embodiment, the optically clear cover is a fused silica glass or a synthetic silica glass (e.g., aluminosilicate glass, borosilicate glass, and soda lime glass).

The optically clear cover may include a hydrophobic surface, a hydrophilic surface, or both. In one embodiment, the optically clear cover provides a hydrophobic surface and a hydrophilic surface. The hydrophilic surface may be positioned directly adjacent to the sheet of porous material. The hydrophobic surface may be positioned opposite the sheet of porous material.

In one embodiment, the optically clear cover layer is removable and replaceable, whereby the sheet of porous material can be replaced, and the biosensor re-used.

To facilitate removal of the cover layer and used sheet of porous material, washing (and drying) of the substrate and cover layer, and re-assembly of the biosensor using a new sheet of porous material, the biosensor may further include (i) a clamping mechanism that compresses the sheet of porous material between the cover layer and the portion of the photonic sensing device or (ii) an adhesive layer connecting portions of the optically clear cover layer directly to the substrate of the photonic sensing device.

The clamping mechanism may include mechanical locks, fasteners, screws, or any other features known in the art for holding together two or more components. In accordance with this embodiment, the optically clear cover layer may include a plurality of through-holes positioned around its perimeter that are designed to align with recesses in the substrate of the corresponding photonic sensing device. The through holes in the optically clear cover layer and the recesses in the substrate may be designed to accept threaded bolts or machine screws positioned around the perimeter of the device (i.e., the substrate and cover layer).

As used herein, “spring clips” are fasteners that grip inserted components through a spring tension. In one embodiment, the clamping mechanism includes spring clips positioned around the perimeter of the biosensor (i.e., a photonic sensing device, a sheet of porous material, and an optically clear cover layer).

In one embodiment, the adhesive layer is suitable to enable reuse of the photonic sensing device, optically clear cover, or both. In accordance with this embodiment, the adhesive layer is in the form of a dual-sided tape or a layer of adhesive applied on the optically clear cover layer. When a cover layer contains adhesive, care should be taken during assembly (or reassembly) to ensure that the sheet of porous material does not interfere with contact between the adhesive layer and the substrate of the photonic sensing device.

A further aspect of the present invention relates to a method of making a biosensor. This method involves providing a photonic sensing device comprising a substrate that contains a three-dimensional structure suitable for producing an optical signal upon exposure to light. This method further involves installing a sheet of porous material onto the substrate, where the sheet covers a portion of the photonic sensing device that contains the three dimensional structure for producing an optical signal, the sheet of porous material comprising one or more capture molecules; and installing an optically clear cover layer over the sheet of porous material, where the sheet of porous material is present between the cover layer and the portion of the photonic sensing device.

In one embodiment, the sheet of substrate, sheet of porous material, and optically clear cover layer are sandwiched together using a clamping mechanism such that the sheet of porous material is static relative to the substrate and optically clear cover layer. In accordance with this embodiment, the sheet of porous material does not make contact with the clamping mechanism.

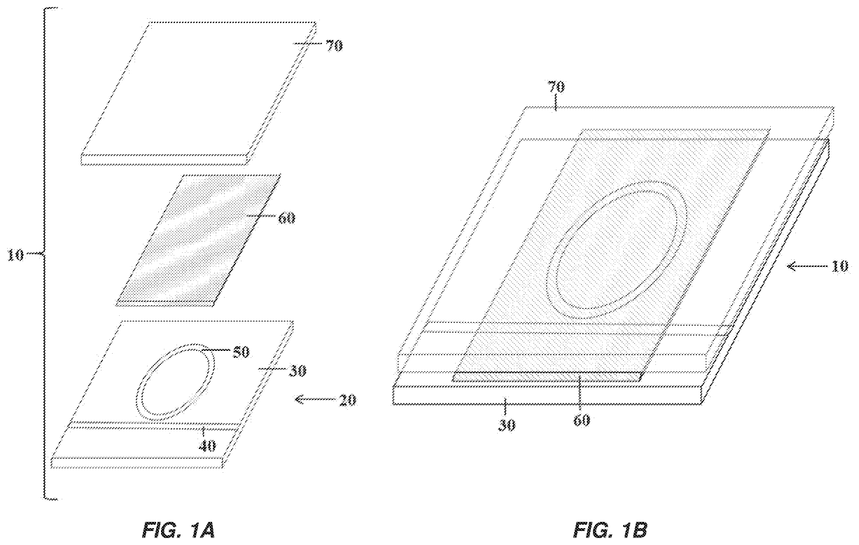

Specific embodiments of the biosensor are described below in connection with FIGS. 1-6. It should be understood, however, that the embodiments illustrated in FIGS. 1-6 are exemplary, and are capable of modification to accommodate different photonic sensing devices of the type described above.

FIGS. 1A-B illustrate biosensor 10, which comprises a photonic chip 20, a sheet of porous material 60, and an optically clear cover layer 70. The photonic chip 20 includes a substrate 30 and formed in the substrate is a bus waveguide 40 optically coupled to a ring resonator 50.

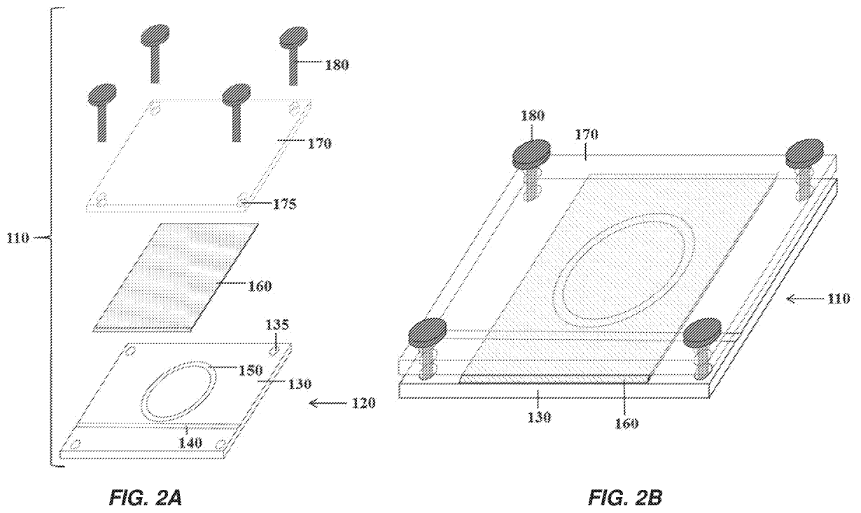

FIGS. 2A-B illustrate biosensor 110, which comprises a photonic chip 120, a sheet of porous material 160, an optically clear cover 170. The photonic chip 120 contains a substrate 130 comprising a bus waveguide 140 optically coupled to a ring resonator 150 and holes 135 positioned at each corner. The optically clear cover 170 comprises holes 175 positioned at each corner, and which are intended to align with the holes 135 in the substrate 130. Together, the holes 135 and 175 accommodate a clamping mechanism 180, which can take the form of a plurality of machine screws if holes 135 are suitably threaded, or mating threaded male and female components.

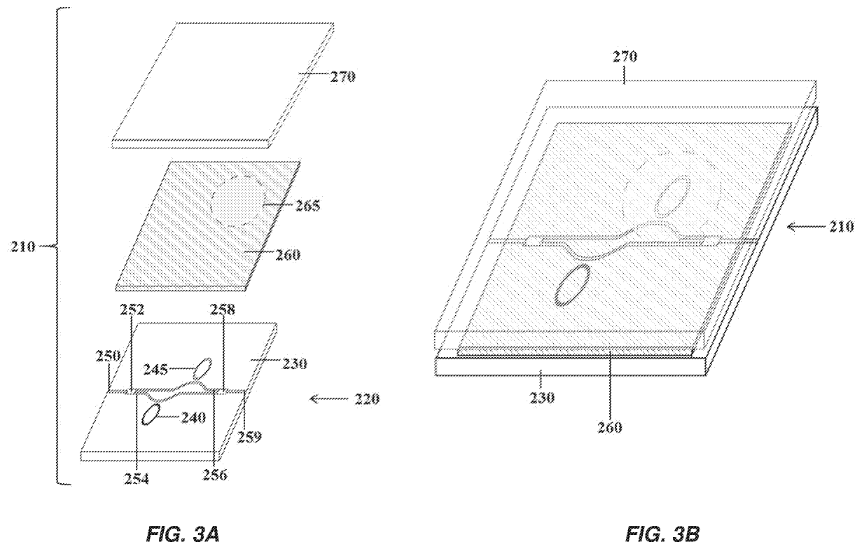

FIG. 3A is an exploded view of a biosensor 210 that includes a photonic chip 220, a sheet of porous material 260, an optically clear cover 270. The photonic chip 220 contains a substrate 230 comprising a ring resonator-coupled Mach-Zehnder interferometer formed in the substrate. The photonic chip 220 comprises an input waveguide 250 that is coupled to a splitter 252, which splits the optical signal between a reference waveguide 254 and a sensing waveguide 256. The reference waveguide 254 is optically coupled to ring resonator 240 and the sensing waveguide 256 is optically coupled to ring resonator 245. The output ends of the reference waveguide 254 and sensing waveguide 256 are joined at coupler 258 to the output waveguide 259. The sheet of porous material 260 includes capture molecule labeled at site 265. In the assembled device shown in FIG. 3B, site 265 overlays ring resonator 245 and its optical coupling to the sensing waveguide 256, but not ring resonator 240 and its optical coupling to reference waveguide 254. Not shown is the clamping mechanism or adhesive layer used to maintain the connection between the cover layer 270 and the photonic chip 220, although both are contemplated for this embodiment.

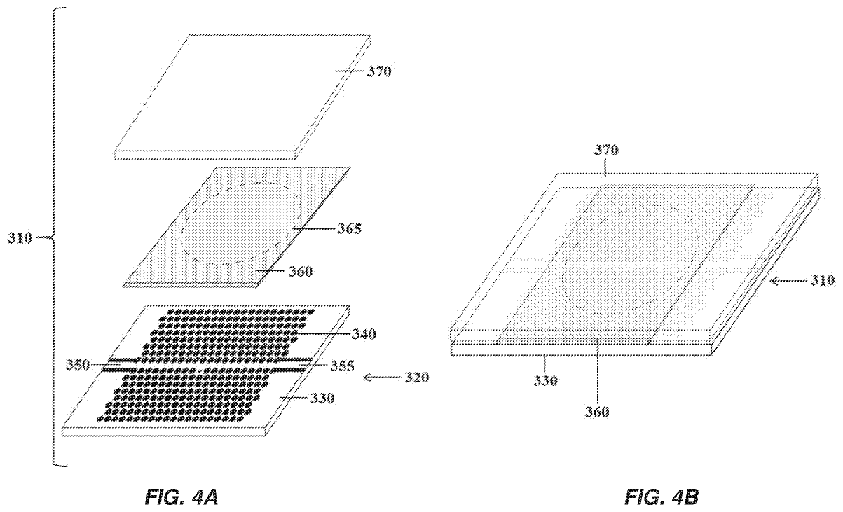

FIG. 4A is an exploded view of a biosensor 310 that includes a photonic chip 320, a sheet of porous material 360, an optically clear cover 370. The photonic chip 320 contains a substrate 330 comprising a photonic crystal array 340 formed in the substrate. The photonic crystal array 340 is composed of a central defect and an ordered array of defects formed about the central defect. Light is coupled into the array by waveguide 350 and light is coupled out of the array by waveguide 355. The sheet of porous material 360 includes capture molecule labeled at site 365. In the assembled device shown in FIG. 4B, site 365 overlays crystal array 340. Not shown is the clamping mechanism or adhesive layer used to maintain the connection between the cover layer 370 and the photonic chip 320, although both are contemplated for this embodiment.

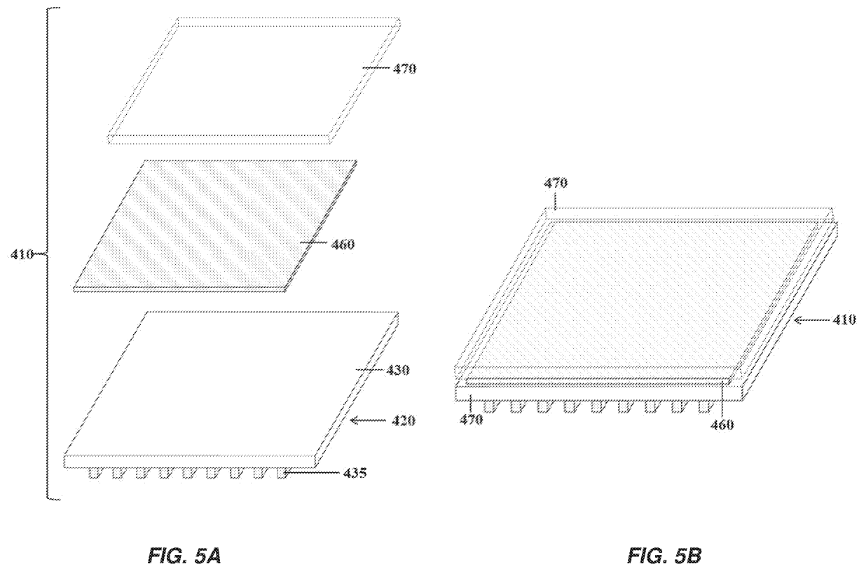

FIG. 5A is an exploded view of a biosensor 410 that includes a photonic chip 420, a sheet of porous material 460, an optically clear cover 470. The photonic chip 420 contains a substrate 430 comprising a diffraction gradient formed therein. The diffraction gradient is comprised of a periodic assembly of ridges 435 (with corresponding adjacent grooves) formed in the substrate. In the assembled device shown in FIG. 4B, the sheet of porous material 460 overlays the substrate 430. Not shown is the clamping mechanism or adhesive layer used to maintain the connection between the cover layer 470 and the photonic chip 420, although both are contemplated for this embodiment.

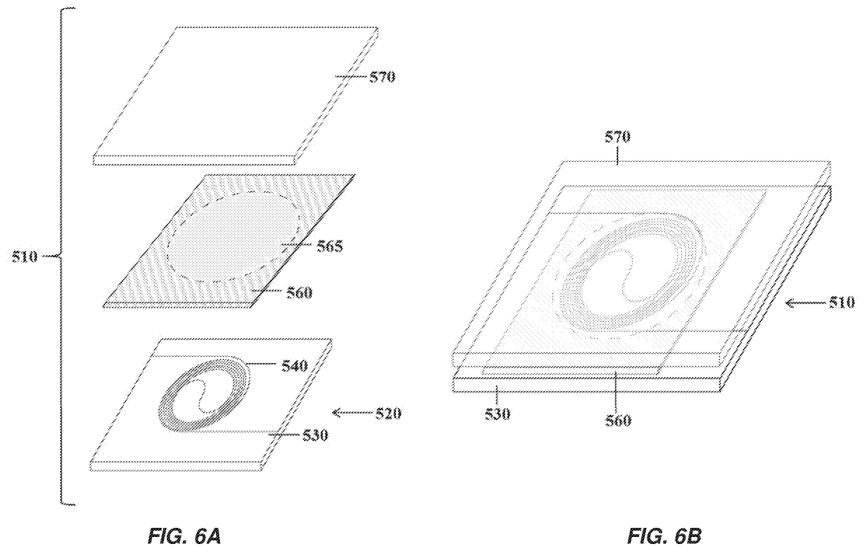

FIG. 6A is an exploded view of a biosensor 510 that includes a photonic chip 520, a sheet of porous material 560, an optically clear cover 570. The photonic chip 520 contains a substrate 530 comprising an Archimedean whispering-gallery spiral waveguide 540 formed in the substrate. This waveguide 540 is characterized by a spiral formation of input and outlet waveguides joined together by a central S-shaped connector. The sheet of porous material 560 includes capture molecule labeled at site 565. In the assembled device shown in FIG. 4B, site 565 overlays spiral waveguide 540. Not shown in is the clamping mechanism or adhesive layer used to maintain the connection between the cover layer 570 and the photonic chip 520, although both are contemplated for this embodiment.

In each of the embodiments shown in FIGS. 1-6, the biosensor and the optically clear cover are roughly the same size and shape, such that the sheet of porous material is only exposed at the edge of the device. Wetting of the sheet of porous material with a liquid sample may be performed by introducing the sample at the edge of the device.

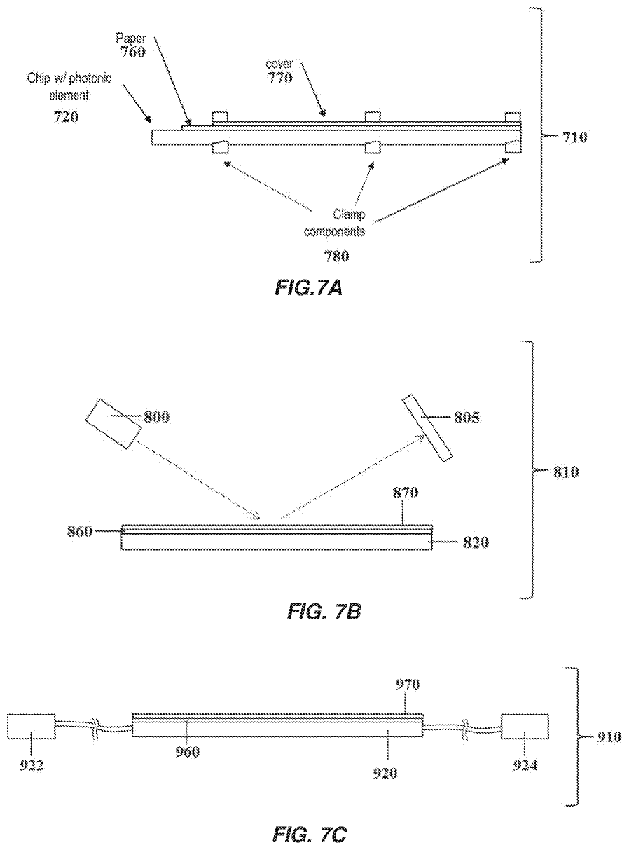

As an alternative construction, shown in FIG. 7A, the photonic chip 720 is longer than the cover 770 in one dimension, and the two components are retained together (with the sheet of porous material 760 compressed therebetween) by the clamping mechanism 780 (three shown). As a consequence, the sheet of porous material 760 is partially exposed along one side of the photonic chip 720. This facilitates wetting of the sheet of porous material with a liquid sample by introducing the sample onto the partially exposed portion of the sheet. The liquid sample (and any target molecule contained therein) will be transported across the sheet of porous material by wicking action.

Another aspect of the present invention relates to a detection device that includes a biosensor as described herein, a light source that illuminates the photonic sensing device; and a photodetection device positioned to measure light emitted by the photonic sensing device.

The light source functions as a source of illumination and may be, for example, an argon, cadmium, helium, or nitrogen laser and accompanying optics positioned to illuminate the biosensor and the detector. The light source may be a laser or broadband light source optionally with a filter. In one embodiment, the light source is a continuous wave light source. In accordance with this embodiment, the slight source is a light emitting diode (“LED”). A skilled scientist will appreciate that different LEDs cover different spectral ranges from about 250 to 1,500 nm. Additional suitable continuous wave light sources include, but are not limited to, Xenon arc lamps, mercury arc lamps, deuterium lamps, tungsten lamps, diode lasers, argon ion lasers, helium-neon lasers, and krypton lasers.

The detection device may further comprise one or both of a waveguide that couples light from the light source into the photonic sensing device and a waveguide that couples light from the photonic sensing device into the photodetection device.

The detector is positioned to capture photoluminescent emissions from the biosensor and to detect changes in photoluminescent emissions from the biosensor. Exemplary detectors include, without limitation, a charge coupled device, spectrophotometer, photodiode array, photomultiplier tube array, or active pixel sensor array. In one embodiment, the photodetection device is a spectrophotometer, photodiode array, photomultiplier tube array, charge-coupled device (“CCD”) sensor, complementary metal-oxide semiconductor (“CMOS”) sensor, or active pixel sensor array.

With reference now to FIG. 7B, a side-elevational view of a detector 810 is illustrated. The detector 810 includes biosensor (with substrate 820, sheet of porous material 860, and optically clear cover layer 870), a light source 800, and a photodetection device 805. Light directed onto the surface of the substrate is reflected from the same, and then measured by detector 805. Changes in the reflected light before and after exposure of the device to a sample can be detected.

With reference now to FIG. 7C, a side-elevational view of a detector 910 is illustrated. The detector 910 includes biosensor (with substrate 920, sheet of porous material 960, and optically clear cover layer 970), a light source 922, and a photodetection device 924. An optical waveguide is used to couple light from the light source to the biosensor (which has an integral input waveguide on the surface of the substrate), and an optical waveguide is used to couple light from the biosensor (specifically, an integral output waveguide on the surface of the substrate) to the detector. Changes in the output light before and after exposure of the device to a sample can be detected.

Yet another aspect of the present invention relates to a method of detecting a biological molecule. This method involves providing a biosensor according to the present invention, introducing a liquid sample into contact with the sheet of porous material; and measuring a change in the light emitted by the photonic sensing device, where the change in the light emitted by the photonic sensing device indicates the binding of the biological molecule by the one or more capture molecules.

Without being bound by theory, when the biosensor includes a ring resonator, wavelengths of light that are exactly equal to the circumference of the ring resonator will become trapped and resonate within the ring, while all other wavelengths of light will leave the ring resonator and be detected by a photonic sensing device. The resonant wavelengths that are trapped in the ring will leave a negative peak in the spectrum of light leaving the ring resonator.

The ring resonator may be made in such a way that a portion of the light energy extends beyond the surface of the ring resonator in the form of an evanescent tail that interacts with the sheet of porous material in the immediate proximity of the ring resonator. The presence of a specific analyte bound by the one or more capture molecules in the sheet of porous material may change the index of refraction and, therefore, change the resonant wavelengths in the ring resonator. The resonant wavelengths will shift proportionally higher as more of the analyte is captured above the ring resonator in the sheet of porous material. This shift in the wavelength is detected by the photonic sensing device as a shift in the negative peak in the spectrum of light leaving the ring resonator. Thus, negative peaks in the intensity of light indicate the resonant wavelengths, and the shift in the wavelengths of the negative peaks indicate a change in the refractive index above the ring cluster, which in turn is proportional to the mass that has bound to the capture molecule above the cluster. In one embodiment, the change in light emitted is measured as a shift in the wavelength of light detected by the photonic sensing device.

As used herein, “biological molecule” refers to molecules derived from, or used with a biological system. The term includes, but is not limited to, biological macromolecules, such as proteins, peptides, carbohydrates, metabolites, polysaccharides, nucleic acids and small organic molecules. The biological marker may be a disease marker.

In one embodiment, the liquid sample is from a subject. As used herein, an “individual” or a “subject” can be any living organism, including humans and other mammals. As used herein, the term “subject” is not limited to a specific species or sample type. For example, the term “subject” may refer to a patient, and frequently a human patient (more specifically, a female human patient or a male human patient). However, this term is not limited to humans and thus encompasses a variety of mammalian or other species. In one embodiment, the subject can be a mammal or a cell, a tissue, an organ or a part of the mammal. Mammals include any of the mammalian class of species, preferably human (including humans, human subjects, or human patients). Mammals include, but are not limited to, farm animals, sport animals, pets, primates, horses, dogs, cats, mice and rats.

As used herein, the term “sample” refers to anything which may contain an analyte (e.g., a biological molecule) for which an analyte assay is desired. As used herein, a “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as saliva, urine, blood, plasma, serum, semen, stool, sputum, cerebrospinal fluid, synovial fluid, sweat, tears, mucus, amniotic fluid, vaginal secretions, tissue and organ samples from animals and plants and processed samples derived therefrom. Examples of biological tissues also include organs, tumors, lymph nodes, arteries and individual cell(s). In one embodiment, the liquid sample is a biological sample.

The biological molecule may include, without limitation, a protein (including without limitation enzymes, antibodies or fragments thereof), glycoprotein, peptidoglycan, carbohydrate, lipoprotein, a lipoteichoic acid, lipid A, phosphate, nucleic acid expressed by a pathogens (e.g., bacteria, viruses, multicellular fungi, yeasts, protozoans, multicellular parasites, etc.), or organic compound such as a naturally occurring toxin or organic warfare agent, etc. Moreover, the biological sensor can also be used effectively to detect multiple layers of biomolecular interactions, termed “cascade sensing.” Thus, a biological molecule, once bound, becomes a probe for a secondary biological molecule. This can involve detection of small molecule recognition events that take place relatively far from the sheet of the porous material.

In one embodiment, introducing a liquid sample into contact with the sheet of porous material may be carried out by placing the liquid sample directly onto the sheet of porous material (or a portion thereof). Alternatively, the sheet of porous material can be exposed to the liquid sample prior to, preferably immediately prior to, assembly of the biosensor.

The presence of the biological molecule in the liquid sample will dictate the change in the light emitted by the photonic sensing device. The change in the light emitted by the photonic sensing device may generally include changes in any one or more of transmission peak wavelength shift, absorption peak wavelength shift, or refractive index change. To determine whether a change in the light emitted by the photonic sensing device has occurred, a baseline optical measurement may be made prior to exposure to a sample. After exposure to the sample, a second optical measurement may be made and the first and second measurements are compared. Typically, any change will depend on the size of the target to be recognized and its concentration within the sample.

Without being bound by theory, when the photonic sensing device comprises a ring resonator, the presence of the biological molecule in the liquid sample causes a change in the absorption peak wavelength shift, where the magnitude of the change is indicative of the concentration of the biological molecule in the liquid sample.

In one embodiment, the extent of the change in light emitted by the photonic sensing device quantifies the amount of the biological molecule in the liquid sample. Thus, the biological sensor of the present invention is suitable for quantitatively detecting an analyte (e.g., a biological molecule) in the liquid sample.

As used herein, “quantitatively detecting an analyte” means that each of the analytes is determined with a precision, or coefficient of variation (CV), at about 30% or less, at analyte level(s) or concentration(s) that encompasses one or more desired threshold values of the analyte(s), and/or at analyte level(s) or concentration(s) that is below, at about low end, within, at about high end, and/or above one or more desired reference ranges of the analyte(s). In some embodiments, it is often desirable or important to have higher precision, e.g., CV less than 30%, 25%, 20%, 15%, 10%, 9%, 8%, 7%, 6%, 5%, 4%, 3%, 2%, 1%, 0.5%, 0.1%, or smaller. In other embodiments, it is often desirable or important that the analytes are quantified with a desired or required CV at analyte level(s) or concentration(s) that is substantially lower than, at about, or at, and/or substantially higher than the desired or required threshold values of the analyte(s). In still other embodiments, it is often desirable or important that the analytes are quantified with a desired or required CV at analyte level(s) or concentration(s) that is substantially lower than the low end of the reference range(s), that encompasses at least 10%, 20%, 30%, 40%, 50%, 60%, 70%, 80%, 90%, 95%, or the entire reference range(s), and/or that is substantially higher than the high end of the reference range(s).

As used herein, an analyte level or concentration “at about” a threshold value or a particular point, e.g., low or high end, of a reference range, means that the analyte level or concentration is at least within plus or minus 20% of the threshold value or the particular point, e.g., low or high end, of the reference range. In other words, an analyte level or concentration “at about” a threshold value or a particular point of a reference range means that the analyte level or concentration is at from 80% to 120% of the threshold value or a particular point of the reference range. In some embodiments, an analyte level or concentration “at about” a threshold value or a particular point of a reference range means that the analyte level or concentration is at least within plus or minus 15%, 10%, 5%, 4%, 3%, 2%, 1%, or equals to the threshold value or the particular point of the reference range.

As used herein, analyte level or concentration that is “substantially lower than” a threshold value or the low end of a reference range means that the analyte level or concentration is at least within minus 50% of the threshold value or the low end of the reference range. In other words, an analyte level or concentration that is “substantially lower than” the threshold value or the low end of the reference range means that the analyte level or concentration is at least at 50% of the threshold value or the low end of the reference range. In some embodiments, analyte level or concentration that is “substantially lower than” the threshold value or the low end of the reference range means that the analyte level or concentration is at least at 60%, 70%, 80%, 90%, 95%, 96%, 97%, 98%, 99% of the threshold value or the low end of the reference range.

As used herein, analyte level or concentration that is “substantially higher than” a threshold value or the high end of a reference range means that the analyte level or concentration is at least within plus 5 folds of the threshold value or the high end of the reference range. In other words, an analyte level or concentration that is “substantially higher than” the threshold value or the high end of the reference range means that the analyte level or concentration is at 101% to 5 folds of the threshold value or the high end of the reference range. In some embodiments, analyte level or concentration that is “substantially higher than” the threshold value or the high end of the reference range means that the analyte level or concentration is at least at 101%, 102%, 103%, 104%, 105%, 110%, 120%, 130%, 140%, 150%, 2 folds, 3 folds, 4 folds or 5 folds of the threshold value or the high end of the reference range.

As used herein, “threshold value” refers to an analyte level or concentration obtained from samples of desired subjects or population, e.g., values of analyte level or concentration found in normal, clinically healthy individuals, analyte level or concentration found in “diseased” subjects or population, or analyte level or concentration determined previously from samples of desired subjects or population. If a “normal value” is used as a “threshold range,” depending on the particular test, a result can be considered abnormal if the value of the analyte level or concentration is more or less than the normal value. A “threshold value” can be based on calibrated or un-calibrated analyte levels or concentrations.

As used herein, “reference range” refers to a range of analyte level or concentration obtained from samples of a desired subjects or population, e.g., the range of values of analyte level or concentration found in normal, clinically healthy individuals, the range of values of analyte level or concentration found in “diseased” subjects or population, or the range of values of analyte level or concentration determined previously from samples of desired subjects or population. If a “normal range” is used as a “reference range,” a result is considered abnormal if the value of the analyte level or concentration is less than the lower limit of the normal range or is greater than the upper limit. A “reference range” can be based on calibrated or un calibrated analyte levels or concentrations.

In accordance with this aspect of the present invention, the method may further involve determining whether the change in light emitted by the photonic sensing device corresponds to about a threshold value, substantially lower than a threshold value, or substantially higher than a threshold value.

A significant advantage of the disclosed biosensors is that they include a disposable component (the sheet of porous material) and re-usable components (one or more of the cover layer, substrate, and any clamping mechanism). Thus, the optically clear cover layer is removable and replaceable such that the biosensor can be re-assembled and re-used by removing the optically clear cover layer and the sheet of porous material after use of the biosensor, thoroughly washing the a photonic sensing device and (optionally) the optically clear cover layer; and using a new sheet of porous material (and optionally a new clear cover layer) to repeat each of the installing steps to re-assemble the biosensor. Washing of the photonic sensing device can be performed using known rinse agents followed by rinsing in water and dried under inert gas (e.g., nitrogen). Thereafter, the biosensor can be used again for multiple detection cycles, following washing and replacement of the sheet of porous material, as described.

### EXAMPLES

The following examples are intended to exemplify the practice of embodiments of the disclosure but are by no means intended to limit the scope thereof.

### Example 1—Integrated Photonic Paper-Based Sensor

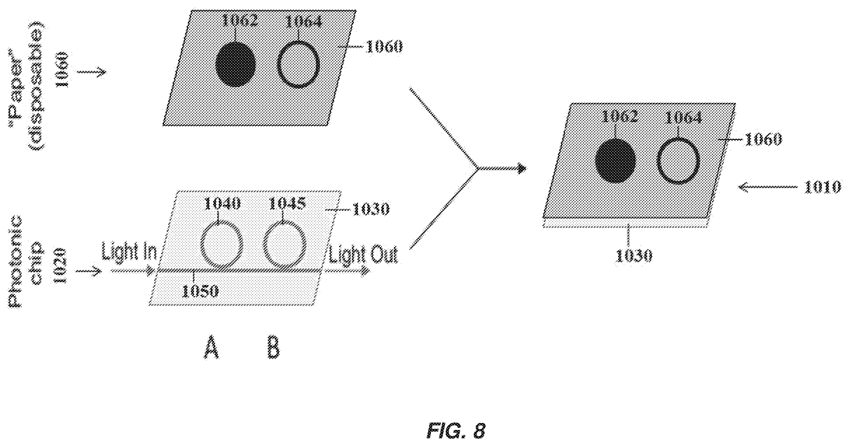

In one implementation of the described biosensor 1010 having a pair of ring resonators 1040, 1045 coupled to a bus waveguide 1050, a capture antibody is spotted onto a nitrocellulose membrane 1060 at one of two locations, 1062, 1064. This may either be via simple adsorption to the paper, or by covalent attachment. The other area 1064 is either functionalized with a control molecule, such as an anti-fluorescein antibody, or is left blank to form a reference zone. The nitrocellulose membrane is placed onto a photonic chip so that the antibody is in register with ring resonator 1045 (FIG. 8). Exposure of the nitrocellulose membrane/photonic “sandwich” to a sample of interest is followed by a wash step after a suitable incubation period.

### Example 2—Integrated Photonic Paper-Based Sensor with Referencing

In another implementation of the described biosensor, a capture antibody is spotted onto a nitrocellulose membrane. The membrane is exposed to a sample, washed, and optionally, dried prior to being placed in contact with a photonic chip. Referencing is provided by either a blank area of the membrane or by comparison with a non-reactive antibody spot such as anti-fluorescein.

In another implementation of the described biosensor, a capture antibody is spotted onto a nitrocellulose membrane. The membrane is used as a fluidic device and a sample is allowed to wick across the active areas. Referencing is provided by either a blank area of the membrane or by comparison with a non-reactive antibody spot such as anti-fluorescein.

### Example 3—Optical Sensor Detection of Nanopure Water and Sucrose Solutions Using an Integrated Photonic Nitrocellulose Membrane-Based Sensors

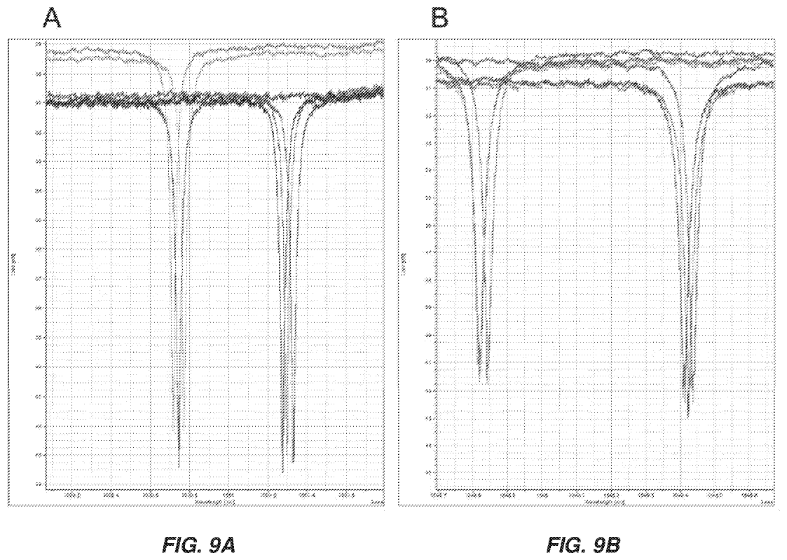

Whether ring resonators function when placed in contact with a nitrocellulose membrane and whether their sensitivity is comparable to the ring resonator alone was evaluated using nanopure water and a sucrose solution. FIGS. 9A-B shows spectra collected for membranes saturated with nanopure water (left clusters) or sucrose solutions (right clusters). In FIG. 9A, nanopure water spectra show clustered resonant wavelengths at 1550.75 nm and 5% sucrose at 1551.30 nm with an average resonant wavelength shift of 0.559 nm (σ=0.013 nm). In FIG. 9B, nanopure water spectra show clustered resonant wavelengths at 1548.85 nm and 5% sucrose at 1549.45 nm with an average resonant wavelength shift of 0.662 nm (σ=0.039 nm).

### Example 4—Optical Sensor Detection of CRPs Using an Integrated Photonic Nitrocellulose Membrane-Based Sensors

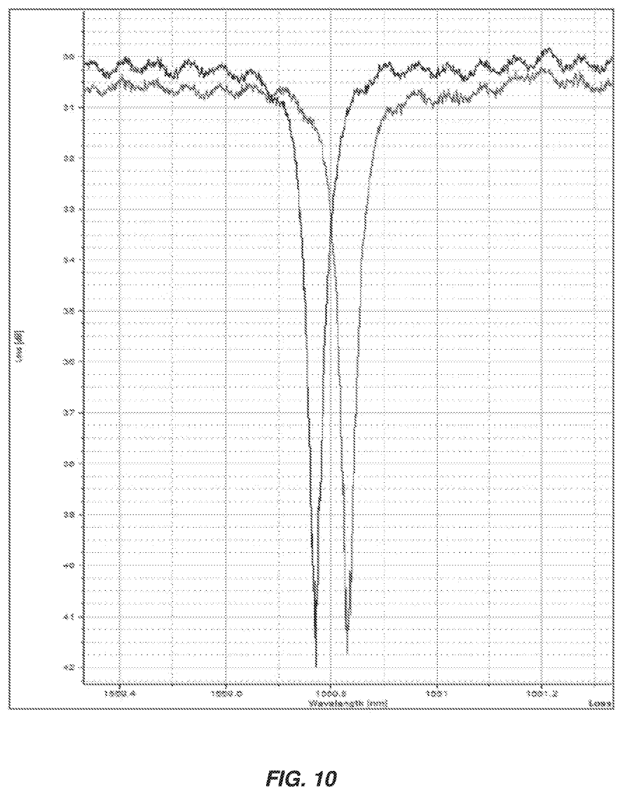

Whether signals due to bulk adsorption of protein on a membrane can be observed was next evaluated. FIG. 10 shows the spectra of nitrocellulose membranes soaked in nanopure water and nitrocellulose membranes with 500 μg/ml α-CRP antibody with 1% BSA block in nanopore water. The resulting resonant wavelength shift is 0.06 nm. This confirms that the sheet of porous material can properly deliver the capture molecule and target molecule, when captured, onto the photonic sensing device in a manner that can alter the resonance behavior to produce a detectable change in output light.

### Example 5—Optical Sensor Detection of BSA Using an Integrated Photonic Nitrocellulose Membrane-Based Sensor

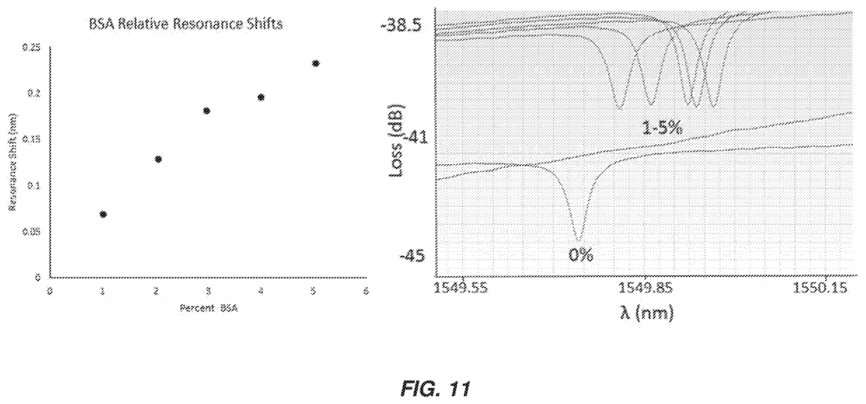

A strip of nitrocellulose was used to deliver protein solution to a ring resonator. A 5-microliter sample of bovine serum albumin (BSA) at different concentrations was applied to a nitrocellulose strip, and allowed to wick across the ring resonator. Concentration-dependent changes in the resonant frequency were observed (FIG. 11). The bulk refractive index sensitivity of the device was measured as 90.8 nm/RIU (via known sucrose solutions). Since chip sensitivities as high as 160 nm/RIU have been measured, the detection sensitivity can likely be substantially enhanced.

### Example 6—Optical Sensor Detection of Human Chorionic Gonadotropin Using Integrated Photonic Nitrocellulose Membrane-Based Sensors

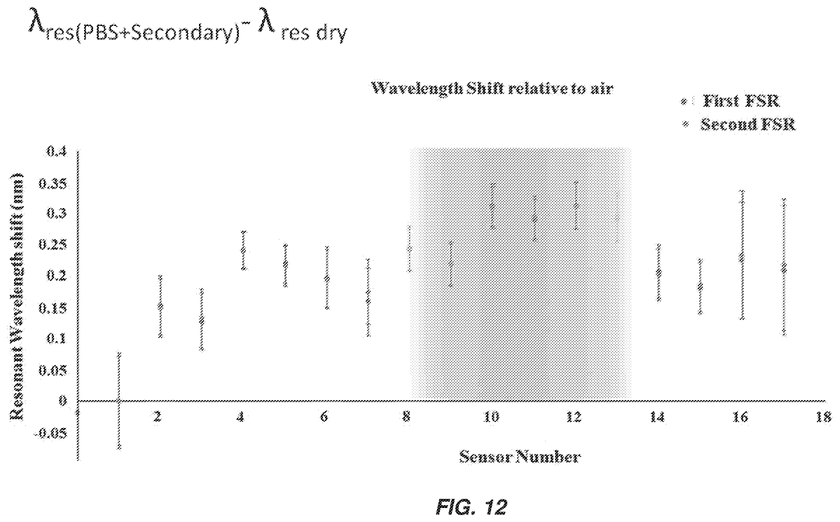

To test whether ring resonators can be used to detect the result of a lateral flow assay, a commercial lateral flow assay for Human Chorionic Gonadotropin was laid across a bank of ring resonators. FIG. 12 shows that stronger shifts were observed for rings under the positive control band (indicated by the shaded area). Two data points representing two separate resonance measurements (FSR, Free Spectral Range) are shown for each ring.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A biosensor comprising:
a photonic sensing device comprising a substrate and, formed on or in the substrate, a three-dimensional structure suitable for producing an optical signal upon exposure to light;
a sheet of porous material covering the three-dimensional structure suitable for producing an optical signal, the sheet of porous material comprising one or more capture molecules; and
an optically clear cover layer connected to the photonic sensing device with the sheet of porous material between the cover layer and a portion of the photonic sensing device that contains the three-dimensional structure.

2. The biosensor according to claim 1 further comprising:
(i) a clamping mechanism that compresses the sheet of porous material between the cover layer and the portion of the photonic sensing device; or
(ii) an adhesive layer connecting portions of the optically clear cover layer directly to the substrate of the photonic sensing device.

3. The biosensor according to claim 1, wherein the photonic sensing device comprises a 2D photonic crystal array, a ring resonator, a Mach-Zehnder interferometer, a toroidal microcavity, a Bragg reflector, a diffraction grating, a plasmonic waveguide, Archimedean whispering-gallery spiral waveguides, or a nanoplasmonic pore.

4. The biosensor according to claim 1, wherein the sheet of porous material comprises polyethylene, polyethylene terephthalate, nylon, glass, polysaccharides, or ceramics.

5. The biosensor according to claim 1, wherein the sheet of porous material is in the form of a paper.

6. The biosensor according to claim 5, wherein the paper has a thickness dimension of less than about 180 microns.

7. The biosensor according to claim 1, wherein the optically clear cover layer is removable and replaceable, whereby the sheet of porous material can be replaced, and the biosensor re-used.

8. The biosensor according to claim 1, wherein the one or more capture molecules is selected from the group of proteins or polypeptides, peptides, nucleic acid molecules, antigens, and small molecules.

9. The biosensor according to claim 1, wherein the one or more capture molecules are covalently attached to the sheet of porous material.

10. The biosensor according to claim 9, wherein the one or more capture molecules comprise a plurality of capture molecules covalently attached to the sheet of porous material at discrete locations.

11. The biosensor according to claim 1, wherein the substrate comprises an inlet for coupling light into, onto, or across the three dimensional structure and an outlet for coupling light that passes from, through, or past the three dimensional structure.

12. A detection device comprising:
a biosensor according to claim 1;
a light source that illuminates the photonic sensing device; and
a photodetection device positioned to measure light emitted by the photonic sensing device.

13. The detection device according to claim 12 further comprising one or both of a waveguide that couples light from the light source into the photonic sensing device and a waveguide that couples light from the photonic sensing device into the photodetection device.

14. The detection device according to claim 12, wherein the light source is a laser or broadband light source optionally with a filter.

15. The detection device according to claim 12, wherein the photodetection device is a spectrophotometer, photodiode array, photomultiplier tube array, charge-coupled device (CCD) sensor, complementary metal-oxide semiconductor (CMOS) sensor, or active pixel sensor array.

16. A method of detecting a biological molecule comprising:
providing a biosensor according to claim 1;
introducing a liquid sample into contact with the sheet of porous material; and
measuring a change in the light emitted by the photonic sensing device, whereby the change in the light emitted by the photonic sensing device indicates the binding of the biological molecule by the one or more capture molecules.

17. The method according to claim 16, wherein the extent of the change in light emitted by the photonic sensing device quantifies the amount of the biological molecule in the liquid sample.

18. The method according to claim 16, wherein the biosensor is reusable upon washing the biosensor and replacing the sheet of porous material onto which the liquid sample is introduced with a second sheet of porous material not previously contacted by a liquid sample.

19. A method of making a biosensor comprising:
providing a photonic sensing device comprising a substrate and, formed on or in the substrate, a three-dimensional structure suitable for producing an optical signal upon exposure to light;
installing a sheet of porous material onto the substrate, whereby the sheet covers a portion of the photonic sensing device that contains the three-dimensional structure for producing an optical signal, the sheet of porous material comprising one or more capture molecules;
installing an optically clear cover layer over the sheet of porous material, whereby the sheet of porous material is present between the cover layer and the portion of the photonic sensing device.

20. The method according to claim 19, wherein the optically clear cover layer is removable and replaceable such that the biosensor can be re-assembled and re-used by:
removing the optically clear cover layer and the sheet of porous material after use of the biosensor,
washing the photonic sensing device; and
using a new sheet of porous material, repeating each of said installing steps.
